(19) United States
(12) Patent Application Publication
(10) Pub. No.: US 2026/0335543 A1
(43) Pub. Date: Oct. 29, 2026

(54) Intravenous Drip Chamber Illumination System with Fluid Level Visualization

(71) Applicant: Martin D. Eason, Waxahachie, TX (US)

(72) Inventor: Martin D. Eason, Waxahachie, TX (US)

(21) Appl. No.: 19/661,013

(22) Filed: Apr. 28, 2026

Related U.S. Application Data
(60) Provisional application No. 63/796,704, filed on Apr. 29, 2025.

(51) Int. Cl. A61M 5/162

(52) U.S. Cl. CPC A61M 5/162; A61M 2205/583; A61M 2205/587; A61M 2205/8206

(57) ABSTRACT

A system for illuminating an intravenous (IV) drip chamber and/or a fluid reservoir to improve visualization of drop formation, flow rate, and fluid volume in low-light clinical environments. The system includes an IV administration set including a spike, a drip chamber, and tubing arranged along a sterile fluid path. An illumination assembly is configured to integrate with or attach to the IV administration set to emit light into IV fluid within the drip chamber to provide visualization in reduced ambient lighting conditions. The illumination assembly includes a housing, an optical guide, and an internal emission interface. Additional and alternative embodiments of the illumination assembly are described herein.

### CROSS REFERENCES TO RELATED APPLICATIONS

This application is related to and claims priority from the following U.S. patent applications. This application claims priority to and the benefit of U.S. Provisional Patent Application No. 63/796,704, filed Apr. 29, 2025, which is incorporated herein by reference in its entirety.

### BACKGROUND OF THE INVENTION

### 1. Field of the Invention

The present invention relates to intravenous (IV) infusion systems and, more specifically, to an integrated system for illuminating IV drip chambers and fluid reservoirs to improve visualization of drop formation, flow rate, and fluid volume in low-light clinical environments.

### 2. Description of the Prior Art

It is generally known in the prior art to provide external intravenous drip lighting devices.

Prior art patent documents include the following:

U.S. Pat. No. 6,050,713 for an Intravenous drip lighting device by inventors O'Donnell et al., filed May 19, 1998, and issued Apr. 18, 2000, is directed to a means of illuminating the drip chamber of intravenous bags during medical processes that are performed in a darkened atmosphere. The apparatus uses an adjustable pole that supports an intravenous bag support that is capable of holding multiple bags and a plate that supports a fiber optic light housing which illuminates all drip chambers adequately and equally and with differing colored light if necessary for distinction. The apparatus is attachable to a procedure table using an adjustable attachment means. The light housing position is fully adjustable in relation to its target.

U.S. Pat. No. 6,877,877 for a Single intravenous drip component illumination device by inventors Rodriguez et al., filed Feb. 13, 2003, and issued Apr. 12, 2005, is directed to a single intravenous drip component illumination device containing a source of light that can illuminate IV bags or bottles, drip chambers and tubing. The directed light of the light source provides adequate lighting for use of the invention in unlit or dimly lit settings with minimum lateral scattering. The device is powered by an external AC or DC power supply and/or by batteries mounted internally to the base of the device.

US Patent Pub. No. 2006/0291211 for an Intravenous drip component illumination device by inventors Rodriguez et al., filed May 26, 2006, and published Dec. 28, 2006, is directed to a single intravenous drip component illumination device containing a source of light that can illuminate IV bags or bottles, drip chambers and tubing. The directed light of the light source provides adequate lighting for use of the invention in unlit or dimly lit settings with minimum lateral scattering. The device is powered by an external AC or DC power supply and/or by batteries mounted internally to the base of the device.

U.S. Pat. No. 5,843,045 for an Infusion illuminator by inventor DuPont, filed Jan. 17, 1997, and issued Dec. 1, 1998, is directed to an I.V. apparatus including a stand, a bag hangably supported at an upper end thereof from the stand and adapted to contain an I.V. solution, a delivery tube for intravenous delivery of the I.V. solution to a patient by a catheter, a drip chamber extending downwardly at an upper end thereof from a lower end of the I.V. bag and connected at a lower end thereof with the delivery tube, a metering device positioned at the lower end of the I.V. bag and operative to deliver I.V. solution in drip fashion from the I.V. bag to the drip chamber, and an illumination device to ensure that the desired drippage flow is taking place. The illumination device comprises a lamp assembly including a housing, batteries positioned within the housing, a light source positioned in the housing and powered by the batteries, a switch controlling the delivery of power from the batteries to the light source, and a clamp for releasably attaching the lamp to the upper end of the drip chamber. With the clamp releasably attached to the upper end of the drip chamber, a rod extending downwardly from the clamp positions the lamp housing in freestanding relation to the drip chamber with the light source positioned proximate to but spaced from the drip chamber so that the light source is operative to illuminate the drip chamber and thereby verify the presence of a drippage flow.

U.S. Pat. No. 6,984,052 for a Drip chamber illumination device by inventor Castillo, filed Oct. 11, 2002, and issued Jan. 10, 2006, is directed to a device for illuminating a drip chamber of an IV bag including a collar with an inner diameter suitable for affixing around the drip chamber and a chemiluminescent container affixed to the interior surface of the collar. The chemiluminescent container serves to emit light therefrom and into the drip chamber. A magnifying lens extends upwardly from the collar so as to magnify an interior of the drip chamber.

U.S. Pat. No. 8,757,828 for a Drip chamber illumination device by inventor Martin, filed Sep. 6, 2012, and issued Jun. 24, 2014, is directed to a drip chamber illumination device including a body and an illumination arm coupled to the body. A light source is disposed on or near the end of the illumination arm wherein the light source may comprise at least one light, but preferably two lights spatially offset that are directed toward a substantially common focal area. The lights may be able to illuminate in different colors. The illumination device further includes a clip attached to the body and configured to removably couple the illumination device to a tube/flush line positioned below a drip chamber. A switch on the device allows a user to selectively provide power from a power source to the lights. The illumination arm may be angled with respect to the body so that the illumination arm may have a substantially vertical orientation when an embodiment of the illumination device is clipped to the tube.

US Patent Pub. No. 2014/0081218 for an Intravenous device having a movable arrangement by inventors Winawer, et al., filed Sep. 4, 2013, and published Mar. 20, 2014, is directed to an exemplary IV system which can include a drip chamber having a movable arrangement configured to mechanically move due to an impact thereof by at least one drop. The movable arrangement can include a spinning arrangement which is configured to spin due to the impact. The spinning arrangement can include a center portion having a plurality of protrusions and can be mounted to the drip chamber using a mounting mechanism.

### SUMMARY OF THE INVENTION

The present invention relates to IV infusion systems.

It is an object of this invention to provide an integrated system for illuminating IV drip chambers and fluid reservoirs to improve visualization of drop formation, flow rate, and fluid volume in low-light clinical environments.

In one embodiment, the present invention includes a system for illuminating an intravenous (IV) administration set, including, the IV administration set including a spike configured to access a fluid reservoir, a flange, and a drip chamber, the drip chamber being arranged along a sterile fluid path extending from the fluid reservoir, and a central illumination cavity integrated into the spike, wherein the central illumination cavity includes a light source, a superior optical window, and an inferior optical window, wherein the light source is operable to be activated and deliver light to the superior optical window and to the inferior optical window, wherein the superior optical window is operable to capture the light and emit the light upwards directly into IV fluid within the fluid reservoir to illuminate the fluid reservoir, and wherein the inferior optical window is operable to capture the light and emit the light downwards directly into IV fluid within the drip chamber to illuminate the drip chamber.

In another embodiment, the present invention includes a method of visually monitoring intravenous (IV) fluid, including, providing an IV administration set including a spike configured to access a fluid reservoir and a drip chamber, the drip chamber being arranged along a sterile fluid path extending from the fluid reservoir, providing central illumination cavity integrated into the spike of the IV administration set, including, a superior optical window, an inferior optical window, a light source, and a power source, activating the light source to deliver light to the superior optical window and to the inferior optical window, wherein the superior optical window is operable to capture the light and emit the light upwards directly into IV fluid within the fluid reservoir to illuminate the fluid reservoir, and wherein the inferior optical window is operable to capture the light and emit the light downwards directly into IV fluid within the drip chamber to illuminate the drip chamber.

In yet another embodiment, the present invention includes a system for illuminating an intravenous (IV) administration set, including, the IV administration set including a spike configured to access a fluid reservoir, a flange, and a drip chamber, the drip chamber being arranged along a sterile fluid path extending from the fluid reservoir, and a central illumination cavity integrated into the spike, wherein the central illumination cavity includes a light source, a superior optical window, and an inferior optical window, wherein the light source is operable to be activated and deliver light to the superior optical window and to the inferior optical window, wherein the superior optical window is operable to capture the light and emit the light upwards directly into IV fluid within the fluid reservoir to illuminate the fluid reservoir, wherein a surface of the superior optical window is curved, faceted, or textured to shape the emitted light, wherein the inferior optical window is operable to capture the light and emit the light downwards directly into IV fluid within the drip chamber to illuminate the drip chamber, and wherein a surface of the inferior optical window is curved, faceted, or textured to shape the emitted light.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 illustrates a sectional side view of the IV drip chamber illumination system, according to one embodiment.

FIG. 2 illustrates an illumination assembly of the system, according to one embodiment.

FIG. 3 illustrates an illumination assembly of the system, according to one embodiment.

FIG. 4 illustrates a top perspective view of the IV drip chamber illumination system, according to one embodiment.

FIG. 5 illustrates an illumination assembly of the system, according to the embodiment shown in FIG. 4.

FIG. 6 illustrates a bottom perspective view of the IV drip chamber illumination system according to the annular light-guide embodiment.

FIG. 7 illustrates a cross-section view of a split-rejoin dual-window spike embodiment of the IV drip chamber illumination system.

FIG. 8 illustrates a perspective view of the rejoin dual-window spike embodiment of the IV drip chamber illumination system.

FIG. 9 illustrates a sectional side view of a spike light guide embodiment of the illumination system.

FIG. 10 illustrates a perspective view of a spike light guide embodiment of the illumination system.

FIG. 11 illustrates a sectional side view of a spike light guide embodiment of the illumination system attached to an IV bag.

FIG. 12 illustrates a perspective view of an alternative embodiment of the illumination system.

FIG. 13 illustrates a perspective view of another alternative embodiment of the illumination system.

FIG. 14 illustrates a cross-section view of an alternative embodiment of the illumination system.

FIG. 15 illustrates a sectional side view of an alternative embodiment of the illumination system.

FIG. 16A illustrates a perspective view of an alternative embodiment of the illumination system.

FIG. 16B illustrates a perspective view of an alternative embodiment of the illumination system.

FIG. 16C illustrates a perspective view of an alternative embodiment of the illumination system.

FIG. 16D illustrates a perspective view of an alternative embodiment of the illumination system.

FIG. 17 illustrates a perspective view of an alternative embodiment of the illumination system.

FIG. 18A illustrates a side view of alternative embodiment of a spike of the IV administration set of the illumination system.

FIG. 18B illustrates a side view of alternative embodiment of a spike of the IV administration set of the illumination system.

FIG. 18C illustrates a side view of alternative embodiment of a spike of the IV administration set of the illumination system.

### DETAILED DESCRIPTION

The present invention is generally directed to an integrated system for illuminating IV drip chambers and fluid reservoirs to improve visualization of drop formation, flow rate, and fluid volume in low-light clinical environments.

In one embodiment, the present invention includes a system for illuminating an intravenous (IV) administration set, including, the IV administration set including a spike configured to access a fluid reservoir, a flange, and a drip chamber, the drip chamber being arranged along a sterile fluid path extending from the fluid reservoir, and a central illumination cavity integrated into the spike, wherein the central illumination cavity includes a light source, a superior optical window, and an inferior optical window, wherein the light source is operable to be activated and deliver light to the superior optical window and to the inferior optical window, wherein the superior optical window is operable to capture the light and emit the light upwards directly into IV fluid within the fluid reservoir to illuminate the fluid reservoir, wherein the inferior optical window is operable to capture the light and emit the light downwards directly into IV fluid within the drip chamber to illuminate the drip chamber, wherein central illumination cavity is isolated from the sterile fluid path by the superior optical window and the inferior optical window, wherein the light source is a light-emitting diode (LED), wherein the central illumination cavity further includes a power source, wherein the power source includes a battery, wherein a surface of the superior optical window is curved, faceted, or textured to shape the emitted light, wherein a surface of the inferior optical window is curved, faceted, or textured to shape the emitted light, wherein an interior surface of the central illumination cavity includes a reflective coating, wherein an interior surface of the central illumination cavity is molded to include a tapered surface, an elliptical or parabolic contour, or a light diffusion-enhancing texture, and wherein the light source is operable to be activated via a power button, a switch, a pull tab, or an automatic power circuit.

In another embodiment, the present invention includes a method of visually monitoring intravenous (IV) fluid, including, providing an IV administration set including a spike configured to access a fluid reservoir and a drip chamber, the drip chamber being arranged along a sterile fluid path extending from the fluid reservoir, providing central illumination cavity integrated into the spike of the IV administration set, including, a superior optical window, an inferior optical window, a light source, and a power source, activating the light source to deliver light to the superior optical window and to the inferior optical window, wherein the superior optical window is operable to capture the light and emit the light upwards directly into IV fluid within the fluid reservoir to illuminate the fluid reservoir, wherein the inferior optical window is operable to capture the light and emit the light downwards directly into IV fluid within the drip chamber to illuminate the drip chamber, wherein central illumination cavity is isolated from the sterile fluid path by the superior optical window and the inferior optical window, wherein the light source is a light-emitting diode (LED), wherein the power source includes a battery, wherein a surface of the superior optical window is curved, faceted, or textured to shape the emitted light, wherein a surface of the inferior optical window is curved, faceted, or textured to shape the emitted light, and wherein an interior surface of the central illumination cavity includes a reflective coating.

In yet another embodiment, the present invention includes a system for illuminating an intravenous (IV) administration set, including, the IV administration set including a spike configured to access a fluid reservoir, a flange, and a drip chamber, the drip chamber being arranged along a sterile fluid path extending from the fluid reservoir, and a central illumination cavity integrated into the spike, wherein the central illumination cavity includes a light source, a superior optical window, and an inferior optical window, wherein the light source is operable to be activated and deliver light to the superior optical window and to the inferior optical window, wherein the superior optical window is operable to capture the light and emit the light upwards directly into IV fluid within the fluid reservoir to illuminate the fluid reservoir, wherein a surface of the superior optical window is curved, faceted, or textured to shape the emitted light, wherein the inferior optical window is operable to capture the light and emit the light downwards directly into IV fluid within the drip chamber to illuminate the drip chamber, wherein a surface of the inferior optical window is curved, faceted, or textured to shape the emitted light, wherein the light source is a light-emitting diode (LED), wherein the central illumination cavity further includes a power source, wherein the power source includes a battery, and wherein an interior surface of the central illumination cavity includes a reflective coating.

None of the prior art discloses an integrated system for illuminating IV drip chambers and fluid reservoirs to improve visualization of drop formation, flow rate, and fluid volume in low-light clinical environments. The system enables care providers to visually assess both the infusion flow and the remaining fluid volume for most fluids and medications without requiring additional external equipment or interruption of workflow, as typically required by the prior art systems. Additionally, illumination may be directed toward the drip chamber, the fluid reservoir, or both. The structural configuration of the system allows integration into standard IV assemblies and supports clinical use in settings where ambient lighting is reduced to accommodate procedural visualization. The system is advantageously operable to be implemented in various embodiments, including integrated sets, modular attachments, and/or other infusion-compatible designs.

Intravenous infusion therapy is widely used in clinical medicine for volume resuscitation, medication administration, and blood product delivery across operating rooms, intensive care units, emergency departments, transport environments, and general wards. In many of these settings, ambient lighting is intentionally reduced to support patient comfort or to enhance visualization of monitors, endoscopic displays, and other image-guided systems, which in turn makes it difficult to visually monitor conventional IV drip chambers and fluid reservoirs.

Additionally, in many procedural environments, such as laparoscopic and robotic operating rooms, interventional radiology suites, bronchoscopy labs, and/or gastrointestinal endoscopy units, ambient lighting is deliberately reduced to enhance monitor visibility. Under these low-light conditions, standard IV drip chambers are often difficult to visualize, making it challenging for anesthesia providers or procedural nurses to assess ongoing fluid delivery.

This limited visibility affects two critical aspects of infusion management: the ability to track drip rate and the ability to estimate the remaining fluid volume in the IV container. Accurate assessment of both is essential for pacing fluid replacement, especially in procedures of intermediate or extended duration. While an IV stand is typically positioned near the provider, the darkness itself makes fluid level discrimination difficult without auxiliary lighting or manual bag manipulation.

Reliable assessment of IV patency and flow rate often depends on direct visualization of drop formation within a drip chamber and estimation of the remaining volume in the reservoir, typically inferred from the position of the air-fluid boundary in the bag or bottle. When ambient light is low, clinicians commonly resort to improvised auxiliary lighting such as task lamps, headlamps, mobile phones, or flashlights, maneuvers that may require repositioning the IV bag or stand and may be incompatible with sterile fields or confined spaces. These workarounds introduce glare and reflections on the chamber wall, obscure the droplet stream, and delay titration or troubleshooting.

In many of these environments, a limiting factor is not only the total amount of light in the room but also the lack of internal contrast within the drip chamber and within the fluid reservoir. Smooth transparent chamber walls, bright specular reflections from overhead sources, and weak illumination of the fluid column make the air-fluid interface and individual drops difficult to distinguish, even when overall room lighting is adequate for other tasks. Because conventional IV sets are illuminated primarily from outside the chamber, the fluid volume often appears as a dim, low-contrast silhouette against a brighter background rather than as a clearly defined, internally lit column, which hinders reliable visualization of drop formation and the fluid-air boundary.

Existing IV infusion sets are generally passive optical devices that rely on whatever external illumination is available; some accessory clip-on lights and external illumination brackets have been proposed, but these remain physically separate from the disposable set, do not consistently illuminate the fluid column itself, and often fail to provide stable, high-contrast visualization of both the drip chamber and the reservoir in realistic clinical workflows. Accordingly, there is a need for an IV administration set that incorporates an internal light source and integrated optical structures configured to deliver guided illumination into the IV fluid within the drip chamber and, in some embodiments, into fluid within the reservoir, to create a high-contrast, internally illuminated fluid column that makes drop formation, patency, and remaining volume readily visible under a wide range of ambient lighting conditions.

The present invention provides an illumination system to make the IV fluid column the primary source of visible light. By directing light through an internal optical interface, whether from a side-mounted aperture, a chamber-mounted ring, an annular guide, a split-rejoin spike assembly, or a reservoir-only emission interface, the system is operable to generate a bright, high-contrast visualization of fluid movement under a wide range of clinical conditions.

Across all embodiments, the system is designed to support clinical environments where visualization is compromised, including but not limited to, operating rooms, interventional suites, emergency departments, transport settings, procedural imaging suites, and/or field or austere environments. The illumination system is operable to enhance safety and workflow efficiency without requiring additional external equipment or deviation from established IV handling techniques.

Referring now to the drawings in general, the illustrations are for the purpose of describing one or more preferred embodiments of the invention and are not intended to limit the invention thereto.

The present invention provides an IV administration set that integrates an internal illumination system into the spike and/or drip chamber region of a disposable IV set. One or more light sources are structurally integrated with the administration set and are optically coupled to internal optical guides or windows that are configured to capture light and emit it through internal emission interfaces directly into intravenous fluid within the drip chamber and/or into fluid within an associated reservoir.

In one embodiment, an internal emission interface is positioned adjacent to the drip-formation region so that light is emitted into the fluid column within the drip chamber, enhancing visualization of drop formation, flow continuity, and the air-fluid boundary under reduced ambient light conditions. In another embodiment, an internal emission interface is positioned adjacent to the spike so that light is emitted into fluid within the reservoir near the spike tip and toward the reservoir air-fluid meniscus without substantially illuminating the drip chamber. In another embodiment, a single illumination system is operable to provide dual-region emission into both the reservoir and the drip chamber.

The internal optical structures may include, by way of example, a columnar light guide extending along the spike with superior and inferior emission regions, an annular light-guide segment coaxial with and surrounding a central drop former and arranged to emit light radially inward into drops and fluid within the drip chamber, a split-rejoin spike configuration in which superior and inferior optical windows transmit light into reservoir fluid and into fluid entering the drip chamber, or a flange or wall window that provides direct optical coupling between a side-mounted light source cavity and the interior of the drip chamber or reservoir.

The illumination system is designed to remain isolated from the sterile fluid path while providing controlled internal emission into the fluid column or reservoir volume. Illumination is thus generated and guided within the administration set itself, rather than being provided solely by external clip-on lights or other accessory illuminators directed at the outer surface of a conventional drip chamber or reservoir. The resulting internally illuminated fluid regions provide bright, high-contrast depictions of drop formation, flow continuity, and fluid boundaries, improving visualization of patency, flow rate, and remaining volume without requiring separate lighting devices or manipulation of the IV stand in low-light clinical environments.

In certain embodiments, the illumination system further includes superior emission regions oriented toward the reservoir air-fluid meniscus, refractive-index-matching fillers positioned between light source cavity windows and associated optical guides to improve coupling efficiency, and optional wavelength-converting materials or circumferential emitter arrangements configured to enhance perceived contrast and provide substantially uniform internal illumination over a wide range of viewing angles.

### General IV Set Architecture

In each embodiment described herein, the IV administration set includes a spike configured to pierce a fluid reservoir port, a drip chamber arranged downstream of the spike, and flexible tubing extending from the drip chamber to a patient access device such as an IV catheter or needle. The spike and drip chamber are preferably molded from transparent or translucent polymeric materials, such as medical-grade polycarbonate or cyclic olefin copolymer, to permit visual inspection of fluid movement and illuminated regions within the set. During use, the spike is inserted into a bag or bottle, fluid flows through a lumen defined by the spike into the drip chamber, drops form at a drop former within the chamber, and the resulting fluid column exits through the tubing for delivery to the patient. Preferably, the spike is operable to pierce the IV bag.

Across these embodiments, the lumen defined by the spike may be dimensioned to correspond to either a macro drip or a micro drip infusion standard. In practice, these variants differ only in lumen diameter; a single sterile fluid passage is present in any given embodiment, and the illumination and light-guide structures described herein apply irrespective of the selected lumen size.

An illumination assembly is integrated with the spike and/or drip chamber and comprises at least one light source operatively coupled to one or more optical structures. The optical structures are configured to capture light from the light source and to guide and emit the light into IV fluid within the drip chamber and, in certain embodiments, into IV fluid within the reservoir adjacent the spike, thereby forming an internally illuminated fluid column that enhances visualization of drop formation, continuity of flow, and the air-fluid boundary. The light source may be electrically powered (for example, a light-emitting diode (LED), organic light-emitting diode (OLED), or electroluminescent element) or may comprise a self-contained chemiluminescent or phosphorescent emitter activated at the time of use. In fluid-contact implementations, the emitter may be encapsulated within a biocompatible transparent polymer such as medical-grade polycarbonate or cyclic olefin copolymer, optionally over-molded around a chemiluminescent capsule or LED package to form a sealed module that is compatible with standard IV set sterilization processes such as ethylene oxide and, in some cases, gamma irradiation. The encapsulation thickness and material are selected to prevent leaching of internal components while maintaining sufficient optical transmission into the surrounding IV fluid.

As used in herein, an “optical guide” means any optical structure configured to receive light from a light source and convey that light toward an emission region or emission interface. An optical guide may include a discrete light pipe, rod, rib, or ring, but may also be implemented by a single optical window or wall segment that provides direct optical coupling between a light source cavity and the interior of the drip chamber or reservoir.

### Illumination Using a Side-Mounted Housing

In one embodiment, the illumination system is implemented without modifying the sterile fluid pathway of an IV administration set or incorporating internal optical guides. Thus, the spike, drip chamber, and lumen geometry remain consistent with standard intravenous administration sets, and illumination is achieved by positioning a light source within a side-mounted housing on the spike body and directing light into the drip chamber through an optical interface formed at or near the spike flange.

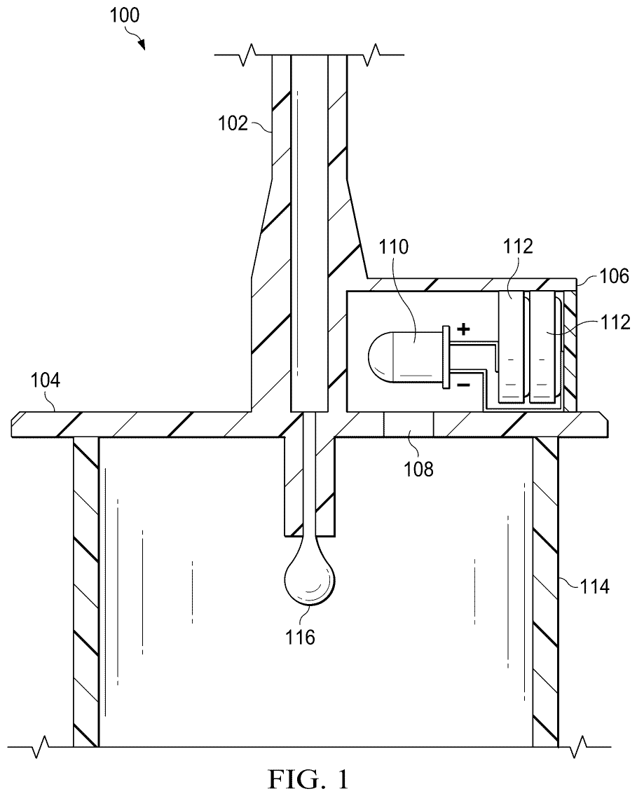

FIG. 1 illustrates a sectional side view of the IV drip chamber illumination system 100, according to this embodiment. The IV drip chamber illumination system 100 includes a spike 102, a flange 104, a side-mounted housing 106, a light guide 108, a light source 110, at least one battery 112 and a drip chamber 114.

In one embodiment, the light source 110 is preferably a miniature LED. In another embodiment, the light source 110 is an LED, an OLED emitter, an electroluminescent element, or a chemiluminescent capsule. The selected light source 110 is operable to emit white light or a specific wavelength band of light tailored to enhance contrast within the drip chamber, wherein the white light source includes a combination of red, green, and blue (RGB) light sources. In one embodiment, the light source 110 may be configured to provide light of a specific wavelength band, including red, orange, yellow, green, cyan, blue, and/or violet. Advantageously, the light source 110 is operable to emit light to illuminate a meniscus level of a drip chamber in a dark environment.

In one embodiment, the light source 110 is configured to provide sufficient internal illumination of the drip chamber and/or reservoir for at least about 6 to 12 hours of clinical use, for example by driving one or more LEDs at currents on the order of a few milliamperes, optionally with duty-cycled operation in the range of about 5% to 50% duty cycle. The resulting internal illumination levels within the drip chamber are selected to provide visually obvious contrast of drops and the air-fluid boundary under reduced ambient light without producing disabling glare for observers.

Light generated within the side-mounted housing 106 is operable to be transmitted into the drip chamber 114 through the light guide 108, such as an optical window formed in the flange 104. In this embodiment, the light source 110 is operable to couple directly into the optical window without the use of internal optical guides, and the shape of the window, including, planar, curved, angled, wide-angle, faceted, or textured, is operable to determine the resulting illumination pattern within the drip chamber 114.

In this embodiment, the light source 110 is positioned within the side-mounted housing 106, wherein the side mounted housing 106 is operable to be molded as part of the spike 102 or added as an integrated molded feature in the spike 102 that does not originally include a housing. In certain commercially available spike architectures, the side-mounted housing 106 corresponds to the location of a conventional vent port; however, in the present embodiment, the side-mounted housing 106 serves solely as a sealed cavity for the illumination assembly and does not provide ventilation or air passage.

Power for the illumination assembly may be provided by one or more coin-cell batteries, thin-film or printed batteries, micro-batteries, a rechargeable cell, or a capacitor charged prior to use. In alternative embodiments, power is delivered from an external generator or controller through sealed electrical contacts or through an inductive-coupling interface positioned on the exterior of the side-mounted housing 106. All power configurations are operable to maintain complete isolation between the illumination assembly and the sterile fluid path. The side-mounted housing 106 may also contain one or more micro-batteries 112, a thin-film power cell, or a compact activation circuit. Advantageously, the light source and power components of the system remain fully isolated from the sterile fluid path and are enclosed within the sealed side-mounted housing 106.

The illumination assembly is operable to be activated via a manual switch, push button, sliding actuator, or pull tab that completes an electrical circuit. Automatic activation mechanisms may also be used, including light-sensitive or motion-sensitive circuits that energize the light source when the administration set is deployed. A timing circuit may intermittently energize the light source to conserve power while maintaining adequate illumination. Duty-cycling or low-current LED drive schemes may be incorporated to extend available operating time during clinical use.

The light guide 108 is also referred to as an optical interface, optical window, or optical aperture herein. Light is delivered into the drip chamber 114 through an optical window or aperture 108 that passes through the flange 104 and provides a direct optical path from the side-mounted housing 106 into the drip chamber 114. The optical window 108 may be centered within the flange 104 or laterally offset within the flange 104, provided it maintains optical coupling to the side-mounted housing 106.

The optical window 108 may take the form of a planar opening, a curved or angled lens surface, a wide-angle refracting interface, or a faceted or geodesic emission structure configured to distribute light into the drip chamber 114. The optical window 108 is formed as part of the spike 102 or flange 104, for example by molding, machining, or post-processing, and remains completely isolated from the sterile fluid path while transmitting light from the side-mounted housing 106 into the drip chamber 114.

In certain embodiments, the light source 110 and associated power components described in this embodiment are not permanently integrated with the disposable administration set but are instead provided as a detachable illumination module/assembly. In these embodiments, the spike 102 or flange 104 defines a mounting structure, receptacle, clip, rail, or keyed interface positioned adjacent the optical window or optical guide 108. The detachable illumination module may be configured to removably couple to this mounting structure such that, when installed, a light-emitting surface of the module is optically aligned with the optical window or guide 108 to deliver light into the drip chamber 114 in the same manner as an integrated light source. Removal or replacement of the illumination module does not alter the sterile fluid pathway, and the disposable administration set retains all optical interfaces required for internal illumination.

Light emitted from the side-mounted housing 106 passes through the light guide 108 and enters the drip chamber 114 as a directed or distributed illumination pattern shaped by the geometry of the light guide 108. This enhances visualization of drop formation 116, flow continuity, and fluid motion in reduced ambient lighting conditions. This embodiment is not configured to transmit light into the fluid reservoir above the spike 102, although other embodiments described herein may provide superior or bidirectional illumination.

Because the sterile fluid path and drip chamber 114 geometry remain unchanged, this embodiment is compatible with existing manufacturing practices for both vented and non-vented spike 102 designs. In architectures where the existing side housing 106 is not contiguous with the flange 104, a molded bridge, thickened wall segment, rib, or structural extension may be used to connect the housing 106 to the flange 104 without affecting the lumen or fluid path. Only minor modifications to the flange 104 are needed to define the optical window 108, advantageously allowing the illumination system to be incorporated with minimal tooling changes.

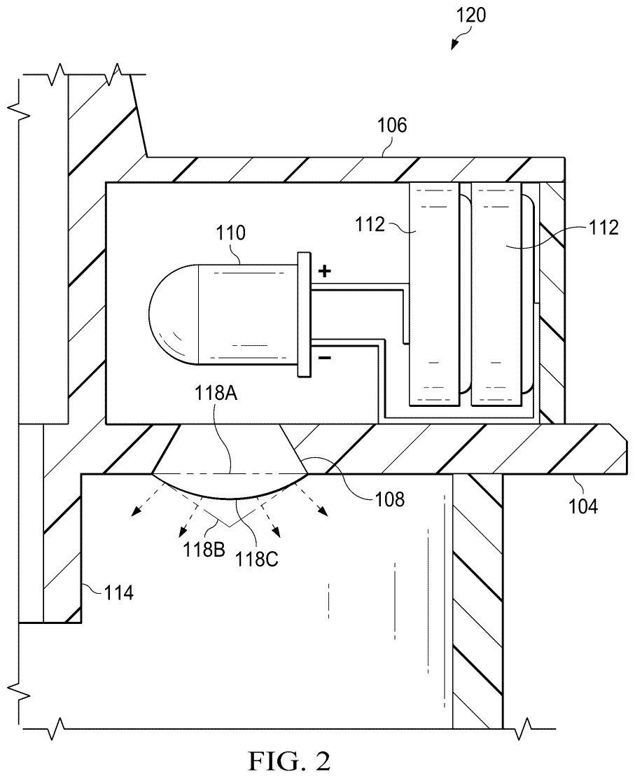

FIG. 2 illustrates an illumination assembly 120 of the system, which includes a side-mounted housing 106, a light guide 108, a light source 110, and at least one battery 112. The illumination assembly 120 is operable to integrate with the spike and/or drip chamber 114. The light guide 108 is operable to be formed at or near the flange 104. In one embodiment, the light guide 108 is operable to be shaped a plurality of different shapes, including angled sides and different shaped bottoms, illustrated as 118A/118B/118C in FIG. 2.

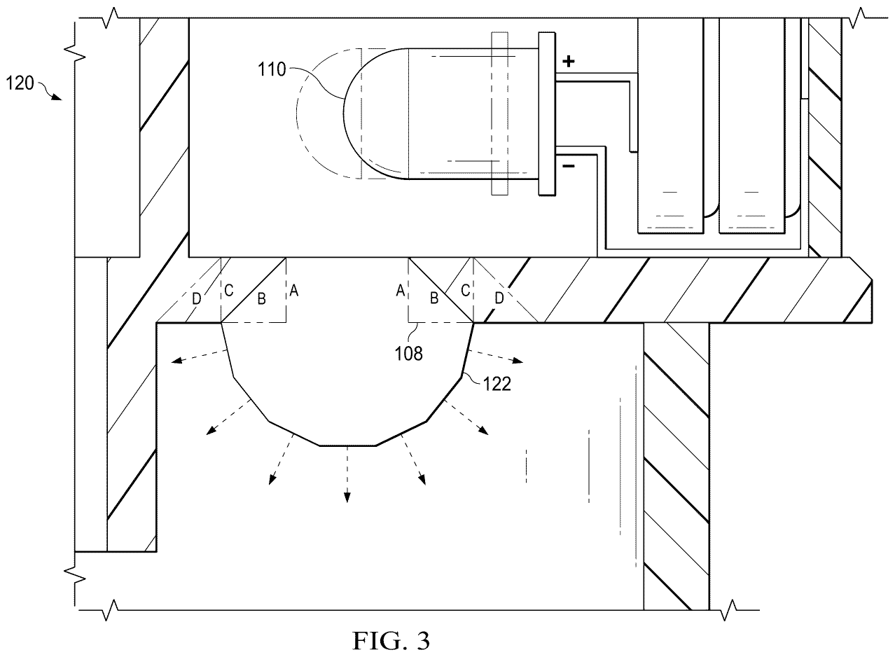

FIG. 3 illustrates an illumination assembly 120 of the system, according to one embodiment. In one embodiment, the light guide 108 includes a lens 122. In one embodiment, the lens 122 is a geodesic dome lens. In one embodiment, the lens 122 includes angled sides, shown as A, B, C, and D in FIG. 3, wherein A is not angled, B is a wide angle, C is a wide aperture, and D is a wide angle and wide aperture. In one embodiment, light rays from a light source 110 are emitted from the bottom of the light guide 108.

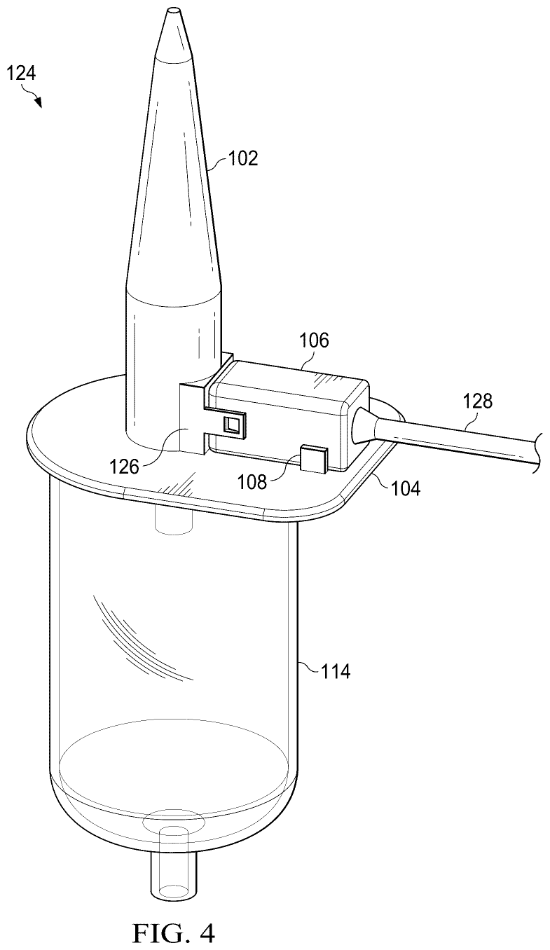

FIG. 4 illustrates a top perspective view of a reusable and clip-on IV drip chamber illumination system 124, according to one embodiment. The IV drip chamber illumination system 124 includes a spike 102, a flange 104, a housing 106, a light guide 108, a drip chamber 114, a clip 126, and a power cord 128. In this embodiment, the illumination system is removable and operable to attach to the spike of an IV administration set using at least one clip 126, wherein the clip 126 attaches the housing 106 to the spike 102, thereby lining up the emitter with the light guide 108.

As shown in FIG. 4, the detachable light source module is positioned relative to the same flange-based optical window as in other embodiments, such that light is coupled into the drip chamber in a manner analogous to the integrated-light embodiments.

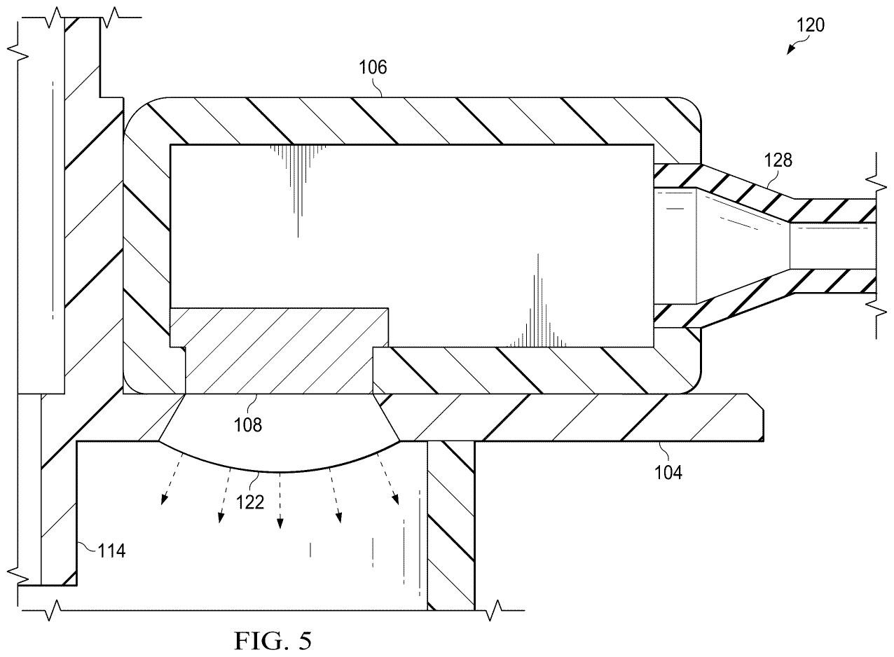

FIG. 5 illustrates an illumination assembly 120 of the system, according to the embodiment shown in FIG. 4. The illumination assembly 120 includes a flange 104, a housing 106, a light guide 108, a lens 122 of the light guide 108, a drip chamber 114, and a power cord 128.

### Wall-Integrated Illumination Ring

In an alternative embodiment, the illumination assembly is incorporated into a circumferential ring or a partial ring that is structurally integrated with the wall of the drip chamber. forming an illumination ring. Alternatively, the ring may be co-molded with the drip chamber body, over molded onto a preformed chamber, or attached as a permanent component through adhesive bonding, solvent welding, ultrasonic welding, or mechanical engagement features. In all implementations, the illumination ring remains external to the sterile fluid path while being manufactured as part of the disposable administration set. In one embodiment, the illumination ring may house one or more light sources such as LEDs, micro-LEDs, OLED emitters, electroluminescent elements, or chemiluminescent capsules, along with an associated power source. In one embodiment, the illumination ring includes an internal cavity or recess configured to protect the illumination components from fluid exposure and to maintain electrical isolation from the fluid path.

During operation, light emitted from the illumination ring is directed radially inward through the transparent or translucent wall of the drip chamber. The drip chamber wall may be formed of unmodified polymer or of polymer that includes molded-in optical features such as planar windows, faceted regions, or localized diffusive textures aligned with the output of the illumination ring. These optical surfaces enhance coupling efficiency and provide uniform illumination of the fluid column and droplet-formation region.

In one embodiment, the illumination ring may extend around the full circumference of the drip chamber or may be limited to one or more arcuate segments positioned to provide the desired illumination pattern. In one embodiment, the ring includes reflective surfaces, internal baffles, or optical shields configured to direct the light primarily inward rather than upward or downward. Advantageously, these features allow the illumination ring to provide bright, localized lighting within the drip chamber without altering the geometry of the spike, flange, or drop former.

The illumination ring may be activated by any of the mechanisms described for the illumination assembly, including manual switches, pull tabs, frangible electrical barriers, ambient-light sensing circuits, or timing circuits that intermittently energize the light source to conserve power. Power may be provided by coin-cell batteries, thin-film printed batteries, rechargeable microcells, or inductive charging elements located within the ring.

The wall-integrated illumination ring embodiment provides internal illumination of the drip chamber without modification of the spike structure or the drip-former assembly, and it is compatible with both macro drip and micro drip chamber geometries. This embodiment may be used alone or in combination with other optional features described herein.

### Wall-Mounted External Illumination Ring

In another alternative embodiment, the illumination assembly is provided as a circumferential or partial-circumferential ring mounted on the exterior surface of the drip chamber. Unlike accessory clip-on devices that illuminate only the outer surface of the chamber, the wall-mounted external illumination ring is operable to form a permanent component of the disposable administration set and may be configured to direct light through the chamber wall into the IV fluid within the drip chamber.

The wall-mounted external illumination ring is operable to be secured to the drip chamber during manufacturing by adhesive bonding, solvent welding, ultrasonic welding, over molding, or mechanical interlocking features formed on the outer surface of the drip chamber. The wall-mounted external illumination ring remains external to the sterile fluid path and may be positioned at any axial location along the drip chamber, including the droplet-formation region, the mid-column region, or the lower portion of the drip chamber.

The wall-mounted external illumination ring is operable to house one or more light sources such as LEDs, micro-LEDs, OLED elements, electroluminescent components, or chemiluminescent capsules, and a power source. In one embodiment, the housing is operable to incorporate an inner surface shaped to act as an optical interface. Suitable geometries for the shape of the inner surface include planar emission windows, curved lenses, prismatic features, or textured surfaces that improve transmission of light through the drip chamber wall and into the fluid column.

In one embodiment, the wall-mounted external illumination ring may further include internal reflective elements, shields, or baffles configured to direct emitted light primarily radially inward. In one embodiment, a portion of the drip chamber wall underlying the wall-mounted external illumination ring may include molded-in optical features such as micro-facets, diffusive patches, or localized thinning to enhance light coupling into the drip chamber interior. These features remain outside the sterile path and do not alter the primary fluid-flow geometry of the drip chamber.

Activation of the wall-mounted external illumination ring may be achieved by the same mechanisms described herein, including manual switches, pull tabs, frangible electrical barriers, ambient-light sensing circuits, or timing circuits that intermittently energize the light source. Power may be supplied by coin-cell batteries, thin-film or printed batteries, rechargeable microcells, or inductive charging components integrated within the ring.

The wall-mounted external illumination ring is operable to provide internal illumination of the drip chamber without modifying the spike, flange, or drop-former structures. Because the light is transmitted directly into the fluid column rather than onto the external chamber wall, this embodiment delivers internal visualization performance similar to that of fully integrated illumination systems while allowing manufacturers to retain conventional drip-chamber tooling.

### Base-Mounted Upward Illumination

In another alternative embodiment, the illumination assembly is operable to be positioned near the base of the drip chamber and configured to direct light upward into the fluid column and droplet-formation region. This base-mounted illumination assembly remains fully external to the sterile fluid path and may be attached to or integrated with the drip chamber outlet structure, the lower drip chamber wall, or the proximal end of the downstream tubing connector.

The base-mounted illumination assembly may take the form of a circumferential or partial-circumferential ring that houses one or more light sources such as LEDs, micro-LEDs, OLED emitters, electroluminescent components, or chemiluminescent capsules, and an associated power source. The housing of the base-mounted illumination assembly is operable to be secured to the base of the drip chamber by adhesive bonding, solvent welding, ultrasonic welding, mechanical interlocks, or over molding, and does not interfere with the normal fluid-flow path through the drip chamber and into the downstream tubing.

During operation, light is emitted upward from the base-mounted illumination assembly through a transparent or translucent portion of the drip chamber floor or lower wall. The upward-directed illumination is operable to be shaped by planar or curved windows, prismatic features, or faceted optical structures positioned above the light source. Advantageously, these features allow the illumination to envelop the droplet-formation zone and the lower portion of the fluid column, providing internal illumination comparable to top-down or side-illumination embodiments.

In one embodiment, the lower drip chamber region is operable to incorporate reflective or refractive geometries that redirect portions of the upward-directed light toward the droplet stream or the upper portion of the drip chamber. The geometries include conical, hemispherical, or compound-curved surfaces; localized wall thinning; diffusive textures; or molded-in microstructures that enhance upward light transmission and provide uniform illumination across the drip chamber interior.

Activation and power supply mechanisms for the base-mounted illumination assembly include those previously described herein, including manual switches, pull tabs, frangible electrical connectors, ambient-light-responsive control circuits, or timing circuits configured to intermittently energize the light source. Power may be provided by coin-cell batteries, thin-film printed batteries, rechargeable microcells, or inductively coupled elements.

The base-mounted illumination assembly is operable to provide internal illumination of the drip chamber without modification of the spike, flange, or drop-former structures, and does not provide superior illumination into the reservoir. The base-mounted configuration is operable to be used with standard macro drip or micro drip chambers and may be combined with other embodiments or optional features described herein.

### Annular Light Guide and Drop Former

In another alternative embodiment, the illumination system includes an annular light-guide structure positioned around a central drop former within the drip chamber. The annular structure is formed from a transparent or translucent material and extends coaxially around the drop former, projecting into the drip chamber interior. The geometry and refractive properties of the annular structure permits light to propagate circumferentially within the ring and to emit through an optical interface toward the fluid within the drip chamber.

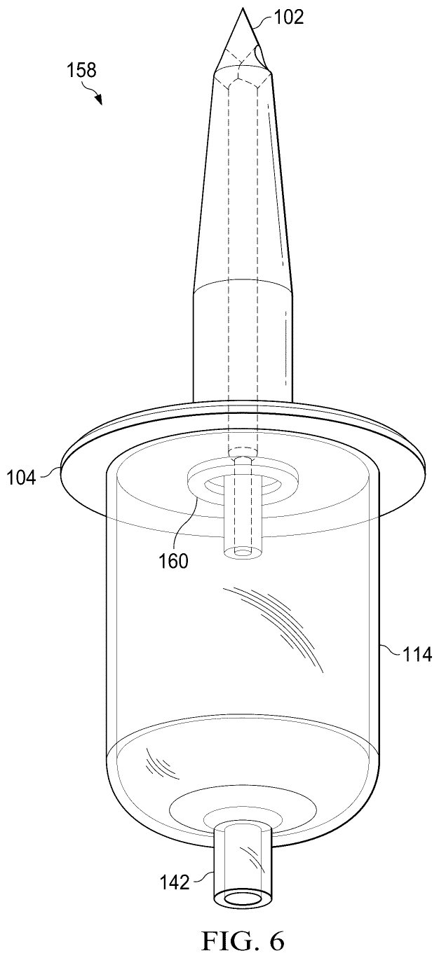

FIG. 6 illustrates a bottom perspective view of the IV drip chamber illumination system 158 according to the annular light-guide embodiment, including a spike 102, a flange 104, a drip chamber 114, a connection element 142 between the drip chamber 114 and IV tubing, and an annular light guide ring 160.

In one embodiment, light is introduced into the annular light-guide structure from a light source located within or adjacent to the spike region, for example through a columnar or radial coupling element, or by direct placement of the light source against an input facet of the annular light-guide structure. In non-fluid-contact implementations, the illumination assembly is isolated from the sterile fluid path by a transparent barrier, whereas in fluid-contact implementations the annular light-guide structure itself may form part of the fluid-contact surface using a biocompatible transparent polymer.

During operation, light emitted from the annular guide is directed radially inward toward the droplet-formation region and the surrounding fluid column. This illumination enhances visualization of individual drops and continuity of flow across typical viewing angles. The inner surface of the annular light-guide structure may include molded micro-facets, diffusive textures, or prismatic elements selected to adjust the intensity and angular distribution of inward-directed light.

In one embodiment, the annular guide includes one or more superior-facing emission interfaces configured to direct a portion of the guided light upward toward the reservoir region. Suitable structures include angled facets, chamfered upper edges, or localized extensions of the annulus that act as secondary emission windows. These optional features allow the annular light-guide structure to illuminate the reservoir meniscus or the fluid immediately above the spike tip while maintaining its primary function of drip-chamber illumination.

In one embodiment, macro drip and micro drip variants are operable to be produced by scaling the annular dimensions and central drop-former geometry. In certain designs, the annular segment and drop former are molded as a single optical cartridge that is inserted into a standard drip-chamber body. The annular light-guide structure may be implemented either as a non-fluid-contact structure separated from the fluid by a transparent barrier or as a fluid-contact optical component fabricated from a biocompatible polymer.

In one embodiment, the annular light-guide structure is compatible with the power and activation mechanisms described herein and may be used alone or in combination with additional optical features disclosed herein.

### Split-Rejoin Dual-Window Spike

In a preferred embodiment, the spike of the IV set is operable to incorporate a split-rejoin lumen geometry arranged around a central illumination cavity to provide illumination of a drip chamber and an IV reservoir bag, thereby advantageously providing a dual-illumination structure. Under this embodiment, the primary lumen entering the spike from the reservoir end is operable to divide into two or more sub-lumens which extend around a central illumination cavity on opposite sides, then rejoin downstream to form a single lumen that empties into the drip chamber. This split-rejoin configuration maintains a continuous sterile fluid path while permitting placement of the illumination assembly within the spike body.

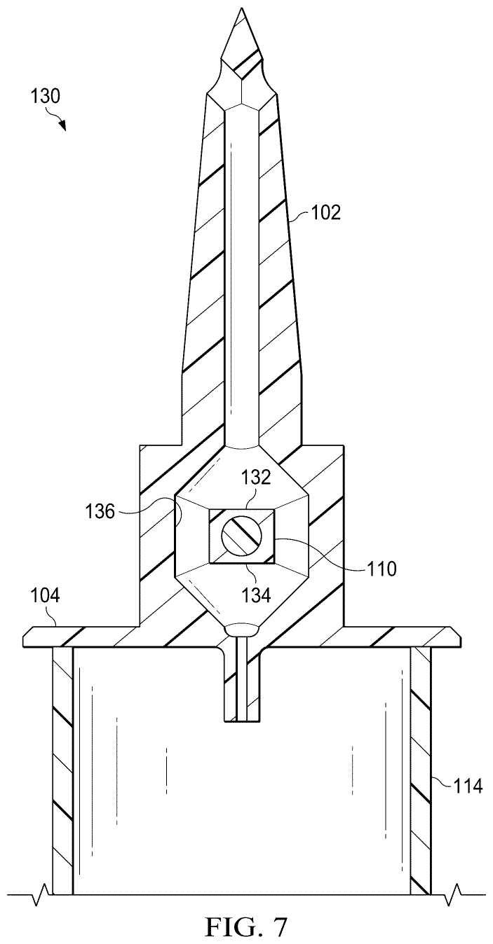

FIG. 7 illustrates a cross-section view of the split-rejoin dual-window spike embodiment 130 of the IV drip chamber illumination system, including a flange 104, a drip chamber 114, and a central illumination cavity 136, which includes a light source 110, a superior optical window 132, and an inferior optical window 134.

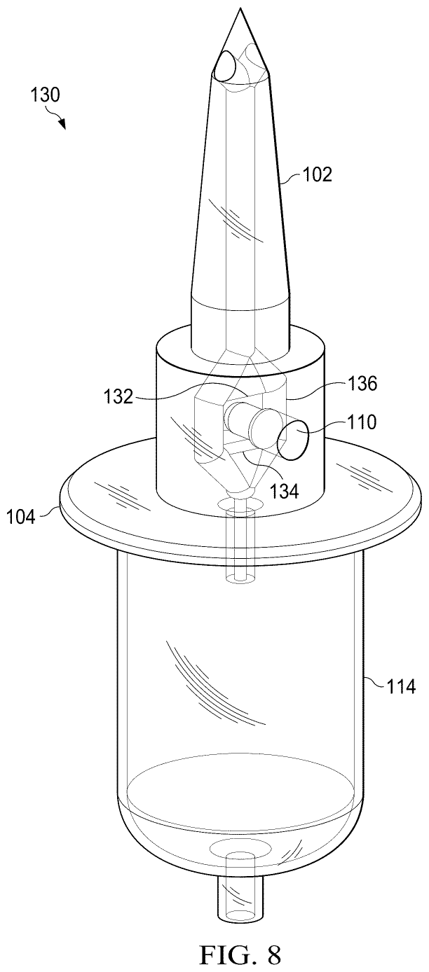

FIG. 8 illustrates a perspective view of the rejoin dual-window spike embodiment 130 of the IV drip chamber illumination system, including a flange 104, a drip chamber 114, and a central illumination cavity 136, which includes a light source 110, a superior optical window 132, and an inferior optical window 134.

In this embodiment, the central illumination cavity 136 houses at least one light source 110, including an LED, an array of LEDs, an OLED element, an electroluminescent emitter, or a chemiluminescent capsule, and an associated power source. In non-fluid-contact implementations, the central illumination cavity 136 is isolated from the sterile fluid path by transparent optical windows 132/134 positioned along the sub-lumen boundaries. In fluid-contact implementations, an encapsulated emitter or sealed optical module is operable to reside directly within the central illumination cavity 136 while maintaining biocompatibility.

At the region where the lumen divides, a superior-facing optical window 132 is formed between the cavity 136 and the portion of the sterile fluid path adjacent the reservoir. Light emitted through this superior-facing window 132 is directed upward into fluid residing within the reservoir or entering the spike from the reservoir. This illumination enhances visibility of the air-fluid boundary or upper reservoir volume when desired.

At the region where the sub-lumens rejoin, an inferior-facing optical window 132 is formed between the cavity 136 and the lumen segment leading into the drip chamber. Light emitted through this inferior-facing window 132 enters that downstream lumen segment and illuminates the fluid as it flows toward and into the drip chamber, including the droplet-formation zone. The window surfaces may be planar, curved, faceted, or textured to shape the emission profile and adjust the distribution of light within the chamber.

The interior surfaces of the illumination cavity 136 may include reflective coatings, molded optical geometries, or compound-curved contours configured to redistribute light toward the superior 132 and inferior 134 optical windows. Suitable geometries include tapered surfaces, elliptical or parabolic contours, facets, and diffusion-enhancing textures that control the balance between upward and downward illumination.

The dimensions of the sub-lumens are selected to preserve flow characteristics similar to those of a conventional spike. The cavity 136 may be sized to accommodate coin-cell batteries, thin-film or printed batteries, rechargeable microcells, or inductively charged power modules. Activation of the illumination assembly may be accomplished through manual switches, pull tabs, frangible barriers, or automatic circuits as described for other embodiments.

This split-rejoin embodiment provides internal illumination of the drip-chamber region and, when configured with the superior window 132, the reservoir region. It remains compatible with standard macro drip and micro drip spike geometries and may be used alone or in combination with other optical structures or illumination features described herein.

### Reservoir-Only Illumination

In another embodiment, the illumination system is operable to be configured to deliver light exclusively to the reservoir region, without providing illumination within the drip chamber. In this embodiment, a light source arrangement is positioned within a cavity formed in the spike body or in a structure immediately adjacent the spike, and an optical guide or window is oriented so that emitted light is directed predominantly into fluid within the reservoir near the spike tip and toward the reservoir air-fluid meniscus, without emitting substantial light into the drip chamber. The spike geometry, reservoir port interface, and sterile fluid lumen remain consistent with standard intravenous spikes, and the illumination components are isolated from the sterile path by transparent barriers or encapsulated emitter housings as described herein. Advantageously, this embodiment is operable to be implemented when enhanced visualization of the reservoir meniscus or upper fluid boundary is desired, but drip-chamber illumination is unnecessary.

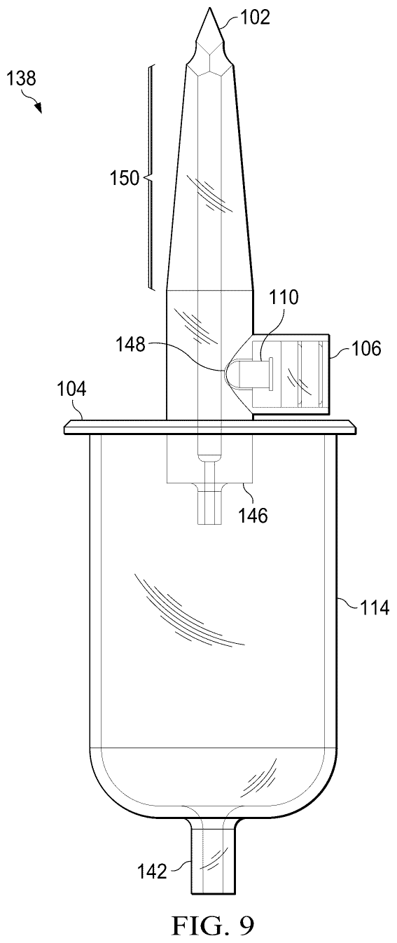

FIG. 9 illustrates a sectional side view of a spike light guide embodiment 138 of the illumination system, including a drip chamber 114, a connection element 142 between the drip chamber and the IV tubing, a drop former 144, a light guide 146, an LED notch 148, a housing 106 containing a light source (LED) and a power source (battery), a superior extension of the light guide into the spike 150 and a spike 102.

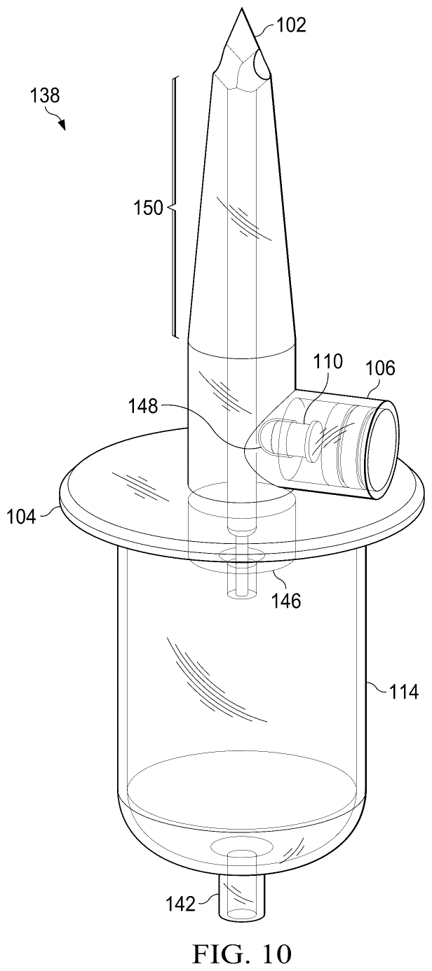

FIG. 10 illustrates a perspective view a spike light guide embodiment 138 of the illumination system, including a drip chamber 114, a connection element 142 between the drip chamber and the IV tubing, a drop former 144, a light guide 146, an LED notch 148, a housing 106 containing a light source (LED) and a power source (battery), a superior extension of the light guide into the spike 150 and a spike 102.

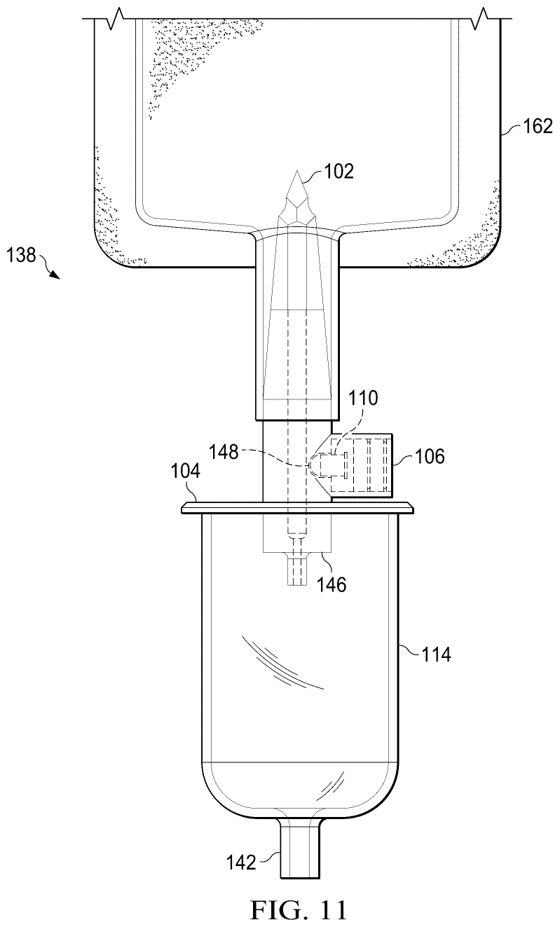

FIG. 11 illustrates an sectional side view of a spike light guide embodiment 138 of the illumination system attached to an IV bag 162. The spike light guide embodiment 138 of the illumination system includes a drip chamber 114, a connection element 142 between the drip chamber and the IV tubing, a drop former 144, a light guide 146, an LED notch 148, a housing 106 containing a light source (LED) and a power source (battery), a superior extension of the light guide into the spike 150 and a spike 102.

In one embodiment, the illumination assembly is operable to reside within or adjacent to the spike structure and may include a light source, including an LED, OLED, electroluminescent element, or chemiluminescent module. In non-fluid-contact implementations, the illumination assembly is isolated from the sterile fluid path by a transparent optical window positioned along the spike wall. In fluid-contact implementations, the emitter may be encapsulated within a sealed, biocompatible housing.

In one embodiment, a superior-facing optical interface is positioned such that light emitted by the illumination assembly is directed upward into the reservoir volume. The superior-facing optical interface is operable to include a spike-tip emission window, a faceted or chamfered spike segment, or a localized light-guide extension that terminates within the fluid volume of the reservoir. The orientation of the interface enables visualization of the air-fluid boundary, including meniscus position, regardless of reservoir orientation.

In one embodiment, the spike is operable to extend slightly farther into the reservoir than a conventional spike to improve light delivery into the open fluid volume beyond the port sleeve. This extension remains compatible with standard reservoir ports and does not alter the sterile fluid path.

This reservoir-only embodiment is operable to incorporate the same activation and power-supply mechanisms described herein and is operable to be combined with diffusive or reflective structures positioned proximate to the spike to intensify upward illumination or to modify the angular distribution of emitted light.

This reservoir-only embodiment is operable to provide illumination of the reservoir region without requiring illumination of the drip chamber. By introducing a superior-directed emission interface located within or adjacent to the spike, it forecloses competitive designs that rely on a reservoir-only spike-mounted light source while avoiding the internal optical guides used in chamber-illuminating embodiments. The architecture is compatible with both macro drip and micro drip spike geometries and are able to be incorporated into any of the configurations described herein.

### Detachable Light Source

In an alternative embodiment, a detachable and reusable light source module is an alternative to the integrated LED/battery light source and is operable to be configured to be used with the same spike/flange/drip-chamber architectures and optical window/light-guide features described herein, such that the primary change is the manner in which the light source is mounted, positioned, and powered.

In certain embodiments, the disposable administration set includes an internal optical coupling region configured to receive light from a reusable external light-supply module. The external module is configured to deliver light into an internal optical structure of the administration set, such as a spike-region light guide, an annular guide surrounding a drop former, or a chamber-mounted internal emission ring, such that light emission into the fluid is defined by the internal optical structure rather than by the external module. In such embodiments, the external module does not directly illuminate any exterior surface of the drip chamber and functions solely as an optical energy source for the internal emission architecture of the administration set.

Light transfer between the external module and the administration set occurs through a defined coupling interface that maintains sterility of the fluid pathway and may include an optical port, alignment key, molded lens element, or combinations thereof to ensure proper optical alignment without external illumination of the drip chamber wall.

This reusable illumination module is structurally and functionally distinct from external clip-on lights, which do not emit light externally through the chamber wall. Instead, external clip-on lights merely inject light directly into an internal light-guiding structure that is part of the administration set.

### Alternative Embodiments

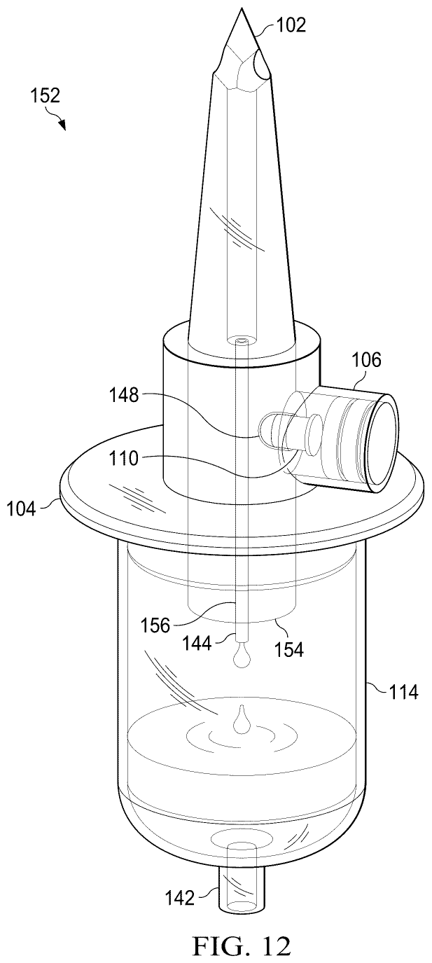

FIG. 12 illustrates a perspective view of an alternative embodiment of the illumination system 152, including a drip chamber 114, a connection element 142 between the drip chamber and the IV tubing, a drop former 144, a light guide 154, a metal micro-lumen 156, an LED notch 148, a housing 106 containing a light source (LED) and a power source (battery). In an alternative embodiment, the illumination system 152 includes a metal micro-lumen 156 in the center made of metal, thereby providing micro drip formation ability. The use of metal advantageously provides enhanced structural integrity over conventional plastic drop formers, thereby providing micro drip formation, which can be implemented in pediatric cases for smaller dosing.

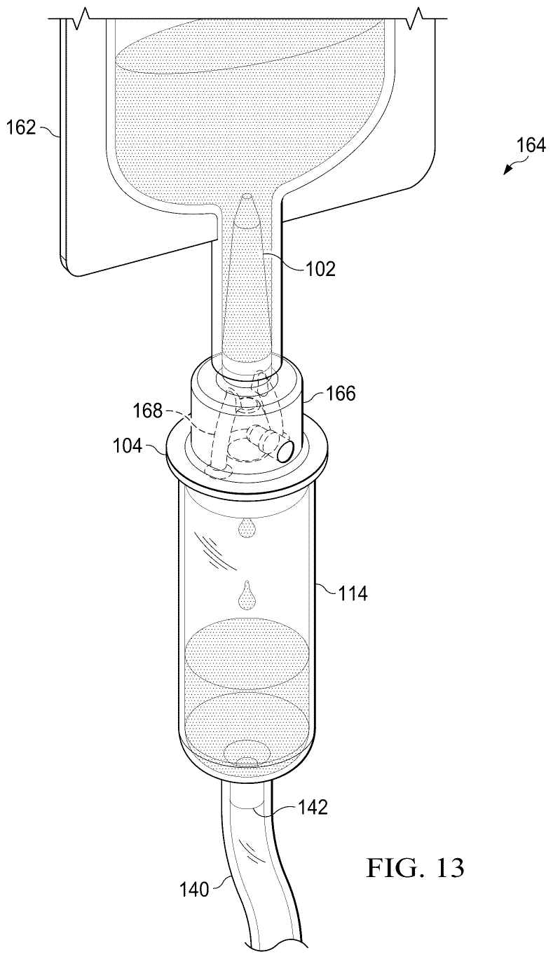

FIG. 13 illustrates a perspective view of another alternative embodiment of the system 164, including, a spike 102, a flange 104, a drip chamber 114, IV tubing 140, a connection element 142, an IV bag 162, a spike base 166, and fluid path structures 168. The bifurcated fluid path structures 168 provide a bifurcated fluid path.

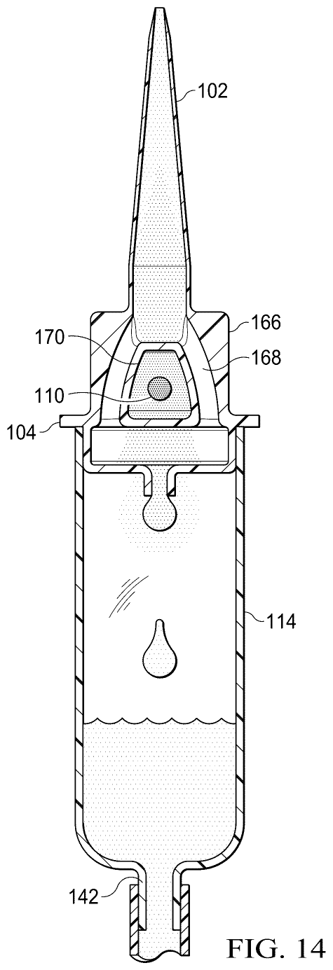

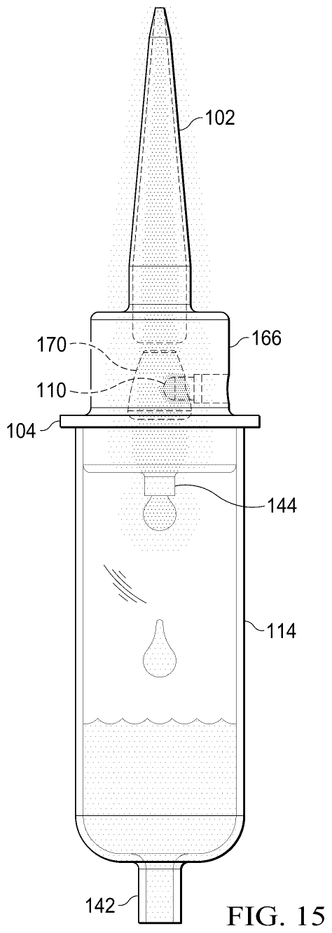

FIG. 14 illustrates a cross-section view of an alternative embodiment, including, a spike 102, a flange 104, a drip chamber 114, a connection element 142, a spike base 166, fluid path structures 168, a light source 110, a housing 170. In one embodiment, the light source 110 is enclosed within the housing 170. FIG. 15 illustrates a sectional side view of this alternative embodiment, including a spike 102, a flange 104, a drip chamber 114, a connection element 142, a drop former 144, a spike base 166, a light source 110, and a housing 170. FIGS. 14-15 illustrate the illumination of both the drip chamber and the spike up to an attached fluid reservoir.

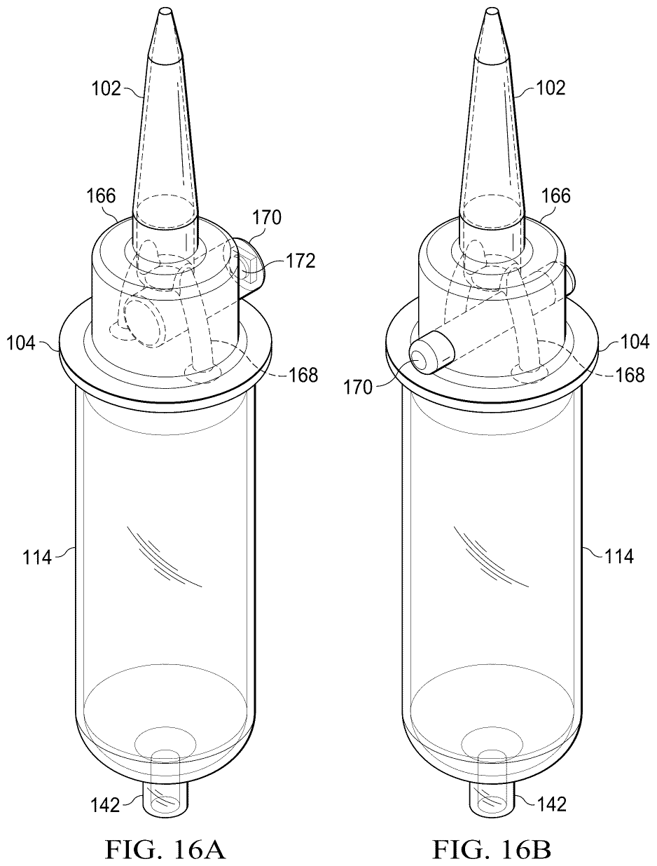

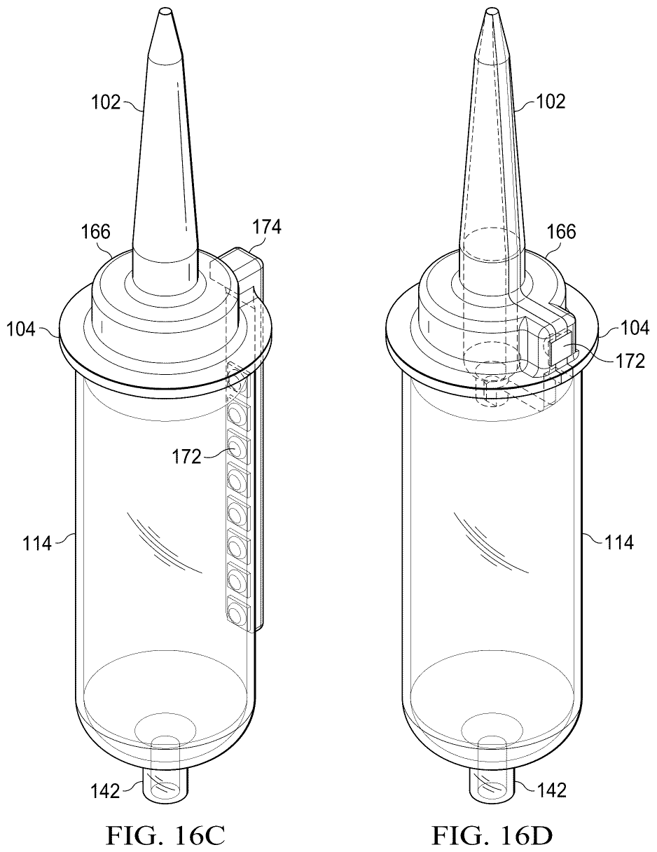

FIGS. 16A-16D illustrate perspective views of alternative embodiments of the system. FIG. 16A illustrates a perspective view of alternative embodiment of the system, including a spike 102, a flange 104, a drip chamber 114, a connection element 142, a spike base 166, fluid path structures 168, an LED light source 172 within a housing 170.

FIG. 16B illustrates an additional perspective view of an additional alternative embodiment, including a spike 102, a flange 104, a drip chamber 114, a connection element 142, a spike base 166, fluid path structures 168, and a housing 170.

FIG. 16C illustrates an additional perspective view of an additional alternative embodiment, including a spike 102, a flange 104, a drip chamber 114, a connection element 142, a spike base 166, an externally applied light source 174 with a plurality of LED light sources 172. Advantageously, the externally applied light source 174 is operable to be attached to the drip chamber 114 such that the plurality of LED light sources 172 are flush against the drip chamber 114, thereby reducing and/or eliminating light reflection on the glass or plastic of the drip chamber 114.

FIG. 16D illustrates an additional perspective view of an additional alternative embodiment of the system, including a spike 102, a flange 104, a drip chamber 114, a connection element 142, a spike base 166, and an LED light source 172.

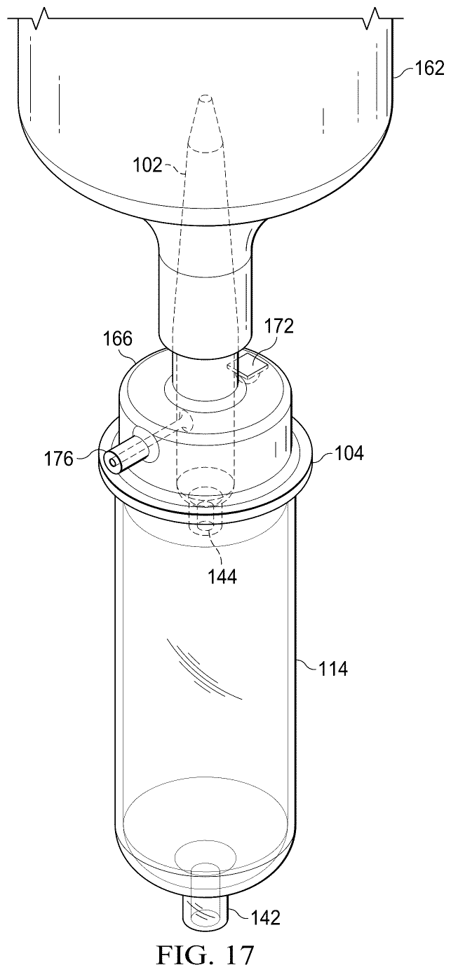

FIG. 17 illustrates a perspective view of an alternative embodiment of the system, including a spike 102, a flange 104, a drip chamber 114, a connection element 142, a drop former 144, a spike base 166, a power button 176, an LED light source 172, and an IV bag 162.

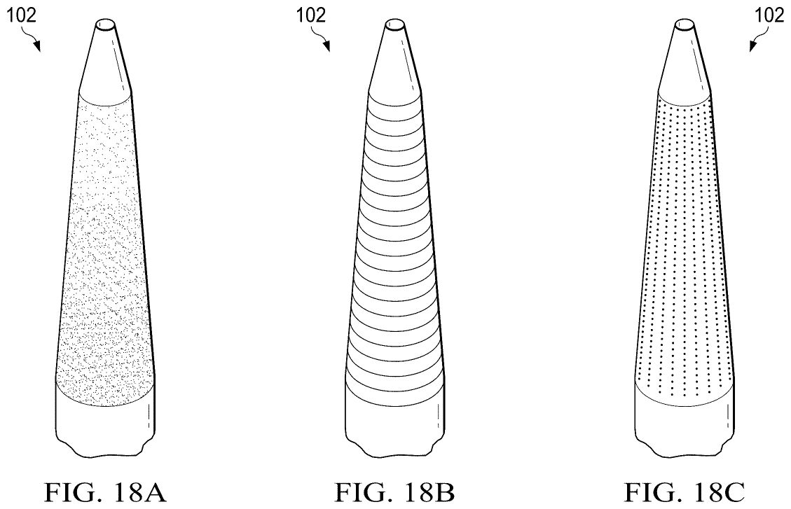

FIGS. 18A-18C illustrate side views of alternative embodiments of a spike 102 of the system, wherein each embodiment shown in FIGS. 18A-18C includes a different textured spike, including a rough texture (FIG. 18A), a ribbed texture (FIG. 18B), or a series or pattern of bumps/divots (FIG. 18C). Advantageously, the textured spike provides enhanced grip for the spike tip when inserted into an IV bag, thus preventing the spike tip from falling out of the IV bag. Additionally, the textured spike provides a pattern of light diffusion into the fluid reservoir.

The various embodiments of the illumination system described herein may be implemented in a variety of configurations that preserve the ability to deliver light into intravenous fluid through an internal optical interface. Differences in geometry, placement, materials, or light-guiding structures do not depart from the scope of the invention.

Illumination may be introduced through a side-mounted optical aperture, an integrated wall-mounted ring, a chamber-base ring, an annular light-guide structure, a split-rejoin spike assembly, or a reservoir-directed emission interface. Any of these features may be used individually or in combination. For example, a device may employ a minimalist aperture for drip-chamber illumination together with a secondary superior emission interface for reservoir visualization.

Optical structures are operable to be implemented as fluid-contact or non-fluid-contact elements. Where non-fluid-contact structures are used, optical windows or transparent barriers are operable to isolate the illumination assembly from the sterile lumen while maintaining optical coupling. Fluid-contact structures are able to be fabricated from biocompatible transparent polymers that form part of the lumen wall or drop-former assembly.

Optical structures may be repositioned, reshaped, reoriented, or dimensioned to modify upward-directed, downward-directed, or radially directed emission.

Light-guiding elements are operable to be straight, tapered, curved, annular, faceted, segmented, or textured. Optical surfaces are operable to incorporate ribs, prisms, diffusive textures, or anti-reflective features to control emission patterns. Reflective or opaque claddings are operable to be continuous or patterned, depending on whether upward, downward, radial, or selective emission is desired. Light-guiding structures may be elongated, shortened, tapered, annular, partially annular, planar, faceted, segmented, or curved.

Emission interfaces may be continuous or discontinuous and may be implemented as apertures, windows, facets, lenses, or molded surface features with smooth, textured, or prismatic finishes.

The illumination assembly of the illumination system is operable to include an integrated power source, including but not limited to, batteries, printed or thin-film batteries, rechargeable microcells, inductive receivers, chemiluminescent sources, electroluminescent elements, and/or any combination thereof. Activation mechanisms for activating the illumination system include, but are not limited to, mechanical switches, pull tabs, frangible circuits, and/or automatic sensing elements. These power and activation features are compatible with any optical geometry described herein.

In certain embodiments, a reusable illumination module is operable to couple light into the disposable administration set through a defined optical port, alignment key, or molded coupling interface. Such modules are operable to inject light into internal optical structures of the disposable set and do not illuminate the drip chamber by external application of light through its wall. These reusable modules are therefore structurally and functionally distinct from clip-on external lights known in the prior art.

In an alternative embodiment, a physical separation element is operable to be included between the housing containing the light source and power supply and the flange or floor of the drip chamber. In this alternative embodiment, light emitted from the light source is operable to be transmitted across the separation element by a transparent optical element, light guide, or optical window structure extending between the housing and the drip chamber, including through aligned apertures or perforations formed in the housing and flange. The optical element is operable to be configured to direct light preferentially into the interior of the drip chamber while limiting lateral or external light emission. In other embodiments, the housing containing the light source is operable to be extended inferiorly to directly contact the flange, thereby eliminating the separation element and forming a continuous optical interface between the light source and the drip chamber.

In an alternative embodiment, a vent channel and hydrophobic filter are included. The hydrophobic filter is operable to be used with rigid containers, such as glass bottles. The vent channel is operable to be positioned adjacent to any illumination assembly described herein, provided that the vent pathway remains fluidly isolated from all electrical and optical components. The presence or omission of a vent structure does not alter the function of the illumination assemblies or their optical interfaces.

In an alternative embodiment, one or more optical components, such as a columnar light-guide segment, an annular segment surrounding a drop former, or a split-rejoin optical window, are operable to be implemented as non-fluid-contact structures separated from the sterile lumen by a transparent barrier, or as fluid-contact optical elements fabricated from biocompatible transparent polymers.

In an alternative embodiment, external surfaces of the drip chamber or spike are operable to include molded diffusive bands, anti-reflective textures, and/or micro-structured regions configured to enhance visibility of the internally illuminated fluid column. These surface treatments do not interfere with the sterile path and serve to improve viewing from oblique angles or under variable ambient light.

In an alternative embodiment, one or more light-emitting or light-guiding components are operable to incorporate a wavelength-converting material, such as a phosphor-doped or quantum-dot-doped polymer layer, positioned along an emission interface so that light of a first spectrum from an LED or other light source is converted to a second spectrum that enhances perceived contrast of the fluid column and air-fluid boundary. Additionally, multiple discrete emitters and/or discrete emission facets are operable to be distributed circumferentially around the droplet-formation region so that the resulting internal illumination is substantially uniform over 360 degrees of viewing angle.

In one embodiment, the illumination assembly is operable to be powered by integrated batteries, printed or thin-film batteries, rechargeable microcells, inductive receivers, chemiluminescent modules, and/or electroluminescent elements. In one embodiment, activation of the illumination assembly is operable to be manual, frangible-tab initiated, or based on automatic sensing mechanisms, such as ambient-light detection or motion-triggered circuits. These activation and power-supply modalities are operable be used with any embodiment described herein.

Any illumination structure described herein is operable to be implemented as a fluid-contact element fabricated from a biocompatible transparent polymer or as a non-fluid-contact structure separated from the sterile lumen by a transparent barrier. Similarly, any embodiment described herein is operable to be scaled for macro drip or micro drip standards and adapted for use with flexible bags, rigid bottles, or other fluid reservoirs.

The illumination assemblies described herein are operable to be integrated with, or optically coupled to, reusable modules that deliver light into internal optical structures of the disposable set through a defined coupling interface while maintaining sterility of the fluid path. These reusable modules do not illuminate the drip chamber by emitting light externally through chamber walls and are structurally distinct from external clip-on lighting accessories.

In an alternative embodiment, the illumination system includes a hybrid configuration of the embodiments described herein. For example, a chamber-mounted ring is operable to be combined with an annular guide, or a split-rejoin spike is operable to be paired with a base-mounted internal ring. Dimensional scaling for macro drip and micro drip variants may be applied to any embodiment without altering the underlying illumination principles.

### Materials and Optics

The illuminated intravenous administration system described herein is suitable for manufacture using standard processes employed in the production of disposable medical devices, including molding, assembly, and sterilization. The individual parts of the system may be produced as single-use sterile products compatible with existing fluid reservoirs such as IV bags and bottles.

Optical components of the illumination system, including apertures, light-guiding segments, annular structures, split-rejoin windows, and/or chamber-mounted rings, are operable to be formed from transparent or translucent medical-grade polymers such as polycarbonate, cyclic olefin copolymer, or equivalent optically clear materials. These polymers may be molded, over molded, cored, or machined to produce the optical interfaces required for internal light delivery. Any portion of the illumination assembly that contacts sterile fluid may be fabricated from biocompatible polymers suitable for intravenous administration.

Non-fluid-contact optical structures are operable to be integrated into the spike, hub, or drip chamber body using the same classes of materials or through incorporation of transparent inserts. Fluid-contact and non-contact variants are both encompassed herein. A transparent barrier separating the illumination assembly from the sterile lumen may be integral to the molded component or implemented as a separate bonded or press-fit element, provided optical coupling to the adjacent guide or emission interface is preserved.

Reflective or opaque claddings are operable to be applied to selected regions of a light-guiding structure to promote internal reflection or to confine emission to designated optical interfaces. Such claddings may consist of pigmented polymers, thin metallic coatings, vapor-deposited reflective layers, or molded-in geometries designed to redirect or contain light. Cladding coverage is operable to be continuous or segmented depending on desired emission patterns.

Emission interfaces, including apertures, facets, textured regions, drop-former surfaces, and superior or inferior windows, are operable to incorporate molded microstructures, diffusive textures, prismatic elements, and/or anti-reflective features to tailor the spatial distribution of emitted light. These surface modifications are operable to improve visibility from oblique viewing angles, intensify illumination at a droplet pinch-off region, and/or modify upward-directed illumination for reservoir visualization. Optical surface geometries are operable to be be smooth, frosted, matte, faceted, or patterned.

When optical coupling is achieved between an illumination cavity and a guide structure, a refractive-index-matching filler is operable to be introduced at the interface to reduce Fresnel losses and improve transmission efficiency. Suitable fillers include optical gels, epoxies, elastomers, and/or low-viscosity transparent polymers compatible with single-use medical devices. The use of such fillers is optional and not required.

All illumination assemblies described herein whether integrated, embedded, or externally coupled, are operable to to withstand standard manufacturing and sterilization processes used for disposable IV sets, including injection molding, gamma sterilization, ethylene oxide sterilization, and/or autoclave-compatible processes.

The various embodiments of the illumination system described herein are configured to direct light through an optical interface to generate visualization of fluid movement in the drip chamber of an IV system, thereby rendering the IV drip chamber the primary source of visible light. All of the embodiments described herein are operable to support clinical environments where visualization is compromised by providing illumination capabilities lacking in the prior art and without requiring additional external equipment or deviation from established IV handling techniques.

### Methods of Use

The setup of an IV administration set is identical to the setup of non-illuminated IV administration sets. The IV administration set is attached to a selected fluid reservoir in accordance with standard practice, such that the spike is inserted into the reservoir port, the drip chamber is primed, and the downstream tubing is filled in the conventional manner.

When illumination is desired, the integrated light source is operable to be activated as described herein, including through a manual switch, a pull-tab or frangible element that completes a circuit, or an automatic control responsive to ambient light, motion, or timing circuitry. Activation does not alter the sterile fluid path or the hydraulic behavior of the IV administration set.

Upon activation, the illumination assembly is operable to direct light through an internal optical interface associated with the selected embodiment. Depending on the configuration, light is operable to be directed toward the drip chamber, toward the reservoir, or toward both regions. The illuminated fluid column, droplet-formation zone, and/or reservoir meniscus becomes visually distinguishable under typical clinical viewing conditions.

Flow rate, drop formation, reservoir volume, or continuity of infusion is operable to be monitored using customary visual assessment techniques. No additional steps, accessories, or external lighting adjustments are required to use the illumination assembly. Thus, clinicians do not have to solely rely on clip-on lights, handheld devices, room lighting adjustments, or other external illumination directed at the exterior surfaces of the drip chamber or reservoir to visualize the fluid, as is provided in the prior art.

Upon completion of the infusion, the administration set is operable to be disposed of in the same manner as other single-use IV sets. Any reusable illumination assemblies are disconnected without contacting the sterile fluid path and may be recharged or otherwise prepared for subsequent use.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A system for illuminating an intravenous (IV) administration set, comprising:
the IV administration set including a spike configured to access a fluid reservoir, a flange, and a drip chamber, the drip chamber being arranged along a sterile fluid path extending from the fluid reservoir; and
a central illumination cavity integrated into the spike, wherein the central illumination cavity includes a light source, a superior optical window, and an inferior optical window;
wherein the light source is operable to be activated and deliver light to the superior optical window and to the inferior optical window;
wherein the superior optical window is operable to capture the light and emit the light upwards directly into IV fluid within the fluid reservoir to illuminate the fluid reservoir; and
wherein the inferior optical window is operable to capture the light and emit the light downwards directly into IV fluid within the drip chamber to illuminate the drip chamber.

2. The system of claim 1, wherein central illumination cavity is isolated from the sterile fluid path by the superior optical window and the inferior optical window.

3. The system of claim 1, wherein the light source is a light-emitting diode (LED).

4. The system of claim 1, wherein the central illumination cavity further includes a power source, wherein the power source includes a battery.

5. The system of claim 1, wherein a surface of the superior optical window is curved, faceted, or textured to shape the emitted light.

6. The system of claim 1, wherein a surface of the inferior optical window is curved, faceted, or textured to shape the emitted light.

7. The system of claim 1, wherein an interior surface of the central illumination cavity includes a reflective coating.

8. The system of claim 1, wherein an interior surface of the central illumination cavity is molded to include a tapered surface, an elliptical or parabolic contour, or a light diffusion-enhancing texture.

9. The system of claim 1, wherein the light source is operable to be activated via a power button, a switch, a pull tab, or an automatic power circuit.

10. A method of visually monitoring intravenous (IV) fluid, comprising:
providing an IV administration set including a spike configured to access a fluid reservoir and a drip chamber, the drip chamber being arranged along a sterile fluid path extending from the fluid reservoir;
providing central illumination cavity integrated into the spike of the IV administration set, comprising:
a superior optical window;
an inferior optical window;
a light source; and
a power source;
activating the light source to deliver light to the superior optical window and to the inferior optical window;
wherein the superior optical window is operable to capture the light and emit the light upwards directly into IV fluid within the fluid reservoir to illuminate the fluid reservoir; and
wherein the inferior optical window is operable to capture the light and emit the light downwards directly into IV fluid within the drip chamber to illuminate the drip chamber.

11. The method of claim 10, wherein central illumination cavity is isolated from the sterile fluid path by the superior optical window and the inferior optical window.

12. The method of claim 10, wherein the light source is a light-emitting diode (LED).

13. The method of claim 10, wherein the power source includes a battery.

14. The method of claim 10, wherein a surface of the superior optical window is curved, faceted, or textured to shape the emitted light.

15. The method of claim 10, wherein a surface of the inferior optical window is curved, faceted, or textured to shape the emitted light.

16. The method of claim 10, wherein an interior surface of the central illumination cavity includes a reflective coating.

17. A system for illuminating an intravenous (IV) administration set, comprising:
the IV administration set including a spike configured to access a fluid reservoir, a flange, and a drip chamber, the drip chamber being arranged along a sterile fluid path extending from the fluid reservoir; and
a central illumination cavity integrated into the spike, wherein the central illumination cavity includes a light source, a superior optical window, and an inferior optical window;
wherein the light source is operable to be activated and deliver light to the superior optical window and to the inferior optical window;
wherein the superior optical window is operable to capture the light and emit the light upwards directly into IV fluid within the fluid reservoir to illuminate the fluid reservoir;
wherein a surface of the superior optical window is curved, faceted, or textured to shape the emitted light;
wherein the inferior optical window is operable to capture the light and emit the light downwards directly into IV fluid within the drip chamber to illuminate the drip chamber; and
wherein a surface of the inferior optical window is curved, faceted, or textured to shape the emitted light.

18. The system of claim 17, wherein the light source is a light-emitting diode (LED).

19. The system of claim 17, wherein the central illumination cavity further includes a power source, wherein the power source includes a battery.

20. The system of claim 17, wherein an interior surface of the central illumination cavity includes a reflective coating.
